(19) United States
(12) Patent Application Publication
(10) Pub. No.: US 2004/0143026 A1
(43) Pub. Date: Jul. 22, 2004

(54) Bioadhesive hydrophilic composition for treatment of mammalian skin

(76) Inventor: Kishore R. Shah, Bridgewater, NJ (US)

Correspondence Address: Nancy A. Bird, 231 Walton Ave, South Orange, NJ, 07079, US

(21) Appl. No.: 10/748,625

(22) Filed: Dec. 27, 2003

Related U.S. Application Data
(60) Provisional application No. 60/437,242, filed on Dec. 31, 2002.

(51) Int. Cl. A61K 9/00; A61K 47/30

(52) U.S. Cl. 514/772.3; 424/400

(57) ABSTRACT

A composition and method for treating mammalian skin by the application of a film-forming composition which is hydrophilic, but water insoluble; said film moisturizing said mammalian skin, providing increased skin retentivity of cosmetic ingredients, and providing sustained release of biologically active agents contained in the composition.

### CROSS-REFERENCE TO OTHER APPLICATIONS

This application claims the benefit of U.S. Provisional Application No. 60/437,242, filed Dec. 31, 2002.

### FIELD OF THE INVENTION

This invention relates to composition, comprising a hydrophilic polystyrene graft copolymer (Copolymer) having bioadhesive properties, for treatment of mammalian skin. This invention also relates to a method of treatment of mammalian skin using the bioadhesive composition. This invention particularly relates to the use of aqueous or non-aqueous formulations containing the Copolymer, with or without biologically active agent, on mammalian skin for cosmetic purposes and/or treatment of dermatological conditions and ailments including skin wounds. The biologically active agent, or any desired cosmetic ingredient, may be provided in either volatile or non-volatile solvents, or as a dispersion in the composition. This invention further relates to transdermal administration of biologically active agents for systemic activity at tissues or organs in the body other than the topical site of application on skin.

### BACKGROUND OF THE INVENTION

Current method of treatment of skin conditions and ailments includes application of formulations, containing therapeutically active ingredient, in the form creams, lotions, solutions, ointments, etc. to the affected area. Some of the indications for which such formulations are used include skin dryness, psoriasis, fungal infections, acne, eczema, dermatitis, itchiness, insect bites, etc. Other uses include application of ultra violet light absorbing agents (sun screen/sun tan). One problem associated with this method of treatment is that the active agent is easily dissipated by rubbing with clothing, washing, or by normal perspiration. Therefore, duration of the agent effectiveness is short. The film forming compositions of the present invention are highly retentive on the skin, and are able to provide long-lasting effectiveness, in both moisturizing, and sustained application of active agent.

Dry skin is one of the most significant cosmetic problems in today's population. It is caused by a reduction in both the water content and the lipid content of the skin. The two main approaches which have been taken in the past to moisturize skin are: (1) to apply compositions containing hygroscopic materials to the skin in order to attract and hold water on the skin's surface, and (2) to apply compositions containing oily materials which form a barrier on the skin and thereby reduce transepidermal water loss through the skin. The first method for moisturization uses hydrophilic molecules which can attract water. Hydrophilic small molecules such as glycerin and glycerin/water mixtures, urea, and propylene glycol are known humectants said to be useful in moisturizing skin. In the latter case, the water level is thought to build up in the skin layer beneath the barrier. Today, the most accepted approach to moisturization involves the use of both methods simultaneously. Most of the moisturizing products in the marketplace today consist of oil-in-water emulsions and creams, water-in-oil emulsions and, to a lesser degree, simply 100% oil formulations. These compositions generally use oils as the main moisturizing ingredient with lesser amounts of humectants. The oils are selected from a large group of commercially available, cosmetically accepted oils, which are generally recognized by the cosmetic industry as having emollient properties.

While these moisturizing products do work, their effects are not long-lasting, i.e., they have to be used repeatedly in order to provide a maximum moisturizing benefit. This is primarily due to the fact that the moisturizing oils do not remain on the skin's surface long enough. Surfactants present in the compositions tend to increase their water-removability.

Another method for moisturization of dry tissue uses an oily substance as the principal ingredient in the form of creams, lotions, gel or salves that are applied to the affected tissue in an attempt to prevent further dehydration of the tissue. They act by placing a water-impermeable hydrophobic barrier over the treated tissue. Petrolatum, mineral oil, lanolin and. isopropyl myristate are examples of hydrophobic materials so used. These preparations are of limited usefulness over a prolonged period of time. In addition, they impart a greasy, sticky feel to the skin and stain clothing.

Oil-soluble acrylate polymers have been used heretofore in sunscreening compositions of the oil or water-in-oil type to reduce removal of the sunscreening agent from the skin by swimming or perspiration. Such compositions are described in U.S. Pat. No. 4,172,122. Other cosmetic compositions containing oil-soluble acrylate polymers are disclosed in U.S. Pat. Nos. 3,911,105; 4,552,755; 4,057,622; 4,057,623; 4,057,624; 4,128,634; 4,128,635; 4,128,636.

J. R. Robinson has disclosed a composition and method for moisturizing epithelial cells. In accordance with this method, the epithelial cells are contacted with an effective moisturizing amount of an aqueous moisturizing composition that contains water, a moisturizing amount of a water-swellable but water-insoluble cross-linked bioadhesive polycarboxylic acid polymer and preferably, a thickening smoothing amount of a consistency-enhancing agent. The bioadhesive polymer is a water-swellable, but water-insoluble, particulate or fibrous, cross-linked carboxy-functional polymer. The polymer is used to contact the epithelial cells of a mammal such as a human.

Hydrophilic, carboxy-functional water-soluble polymers have been used in cosmetic formulations. U.S. Pat. No. 4,863,725 discloses a water-soluble copolymer of glycerol and methacrylic and, namely polyglycerol methacrylate. German Offenlegungsschrift 24 19 046 describes linear and cross-linked polymers containing carboxyl and aldehyde groups as cosmetic compositions.

S. Nayak (U.S. Pat. No. 5,989,535) has disclosed bioadhesive/mucoadhesive composition in a suspension or emulsion form that delivers drugs to the target tissue in a sustained manner. A composition, method of manufacture and its application in treatment of mammalian tissue are disclosed. The composition includes a bioadhesive/mucoadhesive polymer in an emulsion or suspension form along with a treating agent. The treating agent could be as simple as water as in the case of mucoadhesive moisturizing agent. The bioadhesive/mucoadhesive polymer is a water dispersible high molecular weight crosslinked polyacrylic acid copolymer with free carboxylic acid groups further crosslinked with a combination mono, di and polyvalent metal ions, cationic polymers and surfactants.

Topical medications that include biologically active agents, such as corticosteroids, are used for treating skin conditions such as atopic dermatitis, psoriasis and other pathologies of the skin. Current steroid-containing products are available mainly as gels, lotions or ointments that are supplied in tubes or bottles and applied to an affected area of the skin by hand. To enhance the effect of a steroid agent on the skin, it is desirable to have a moisturizing or emollient effect to supplement the curative action of the steroid. Also, it is preferred that an occlusive barrier be applied to the skin during application to enhance the retention and the bioavailability of the steroid.

U.S. Pat. No. 5,874,074 discloses an occlusive or semi-occlusive barrier moisturizing lotion useful for treating pathologies of the skin. The lotion is composed of an oil-in-water emulsion that includes water, one or more emollient, at least one polyhydric alcohol, a water-soluble film-forming barrier polymer, and a therapeutic agent, preferably a steroid such as a corticosteroid. The water-soluble film-forming barrier polymer is selected from a group consisting of vinyl pyrrolidone hompolymers and copolymers. Upon application to the skin, the lotion forms an occlusive or semi-occlusive water soluble polymeric barrier film that retains the therapeutic agent in intimate contact with the surface of the skin, but, being water soluble, can easily come off with water.

Shah (U.S. Pat. No. 5,942,243) has disclosed polymeric mucoadhesive compositions comprising a graft copolymer having a hydrophilic main chain and hydrophobic graft chains. The hydrophobic graft chains consist essentially of polystyrene. The hydrophilic main chain contain monomeric units, wherein at least about 10% by weight based on the total weight of the graft copolymer, which have acidic functionality. Now, it has been found that the said mucoadhesive graft copolymer exhibits a unique combination of tissue moisturizing properties, substantivity on skin, and retention on the skin of bioactive agent formulated with it. Formulations of the graft copolymer in the form of a lotion, cream, gel, or an organic solution, when applied to the skin, form an invisible, bioadherent, hydrophilic but water-insoluble polymeric barrier film on the skin.

### SUMMARY OF THE INVENTION

The present invention is directed to a bioadhesive, hydrophilic polymer film forming composition, comprising a water-insoluble graft copolymer (Copolymer), in the form of a solution, emulsion, dispersion, lotion, cream, gel, film, or petrolatum or wax based preparations for treatment of mammalian skin. This invention particularly relates to the use of aqueous or non-aqueous formulations containing the Copolymer, with or without biologically active agent, on mammalian skin for cosmetic purposes and/or treatment of dermatological conditions and ailments including skin wounds. This invention also relates to a method of treatment of mammalian skin using the bioadhesive polymeric composition. This invention further relates to transdermal administration of biologically active agents for systemic activity at tissues or organs in the body other than the topical site of application on skin.

The graft copolymers suitable for use in this invention include those described in the Shah U.S. Pat. Nos. 5,814,329 and 5,942,243. The graft copolymer has a hydrophilic polymeric main chain and a hydrophobic polymeric side chain. The main chain is comprised of hydrophilic monomeric units, some which have acidic groups. The acidic monomers include, but are not limited to, acrylic and methacrylic acids, 2-acrylamido-2-methyl-propane sulfonic acid, 2-sulfoethyl methacrylate, and vinyl phosphonic acid. The hydrophobic side chain moiety is polystyrene. A full description of the components of the graft copolymer is set forth in U.S. Pat. Nos. 5,814,329 and 5,942,243, the complete disclosures of which are incorporated herein by reference. The sustained release of an active agent from such polymers has been demonstrated in, “Novel In Situ Gelling Liquid Mucoadhesive For Controlled Drug Delivery”, K. R. Shah and William J. Tillman, Poster Presentation Abstracts, American Association of Pharmaceutical Scientists National Meeting (October 2000).

The copolymer film-forming compositions of the present invention are bioadhesive, film forming, and highly skin retentive; attributes that could not be predicted from their mucoadhesive property. They may easily incorporate any acceptable liquid pharmaceutical excipient. In addition, any aqueous, or non-aqueous formulation of a cosmetic additive, incorporating either volatile, or non-volatile solvents, may be incorporated into the film-forming, highly skin retentive compositions of the present invention. Thus they provide a universally applicable vehicle for application of biologically active agents, or cosmetic components, to the skin.

The topical application of any one of the foregoing dosage forms on skin is a method of forming an imperceptible hydrogel film comprising of the Copolymer. Key properties of the formed Copolymer film on skin are its transparency, hydrophilicity, moisture retentiveness, breathability, bioadhesion (high substantivity on skin), and capability to provide sustained release of an active agent, which can be as simple as moisture, over a prolonged period of time. The method of treating skin with a Copolymer dosage form of this invention provides the benefit of a long lasting treatment, which at once enhances user convenience and compliance. Further, by virtue of providing sustained release of an active to skin, the effectiveness of the active may be optimized and undesirable side effects may be decreased.

It is an object of the present invention to provide a film-forming composition for moisturizing mammalian skin, and for the application of biologically active agents, and cosmetic components to the skin. The film provides high skin retentivity, retaining the active agent against the skin, in a water insoluble film providing long lasting, sustained release of the active agent. The film also serves to maintain the cosmetic component in a water insoluble film, providing a long lasting make-up, or treatment. The film forming composition may incorporate volatile or non-volatile solvents, as needed, to incorporate biologically active agents and cosmetic components. In addition, the compositions may incorporate any number of ingredients, and thus may be used to apply a treatment, and to treat the side effects of the treatment. The universally applicable composition will find many uses. These objects, as well as other objects which will become apparent from the discussion that follows, are achieved, in accordance with the present invention which provides a film-forming composition for application to mammalian skin. The composition comprises a) from about 0.3% to about 10% by weight of the total composition, of a graft Copolymer, comprising a hydrophilic polymer main chain comprising monomeric units, some of which have acidic groups, and a hydrophobic polymeric side chain comprising polystyrene; and b) from about 0 to about 75% of water soluble polymer by weight, based on the combined weights of the water soluble polymer and the graft copolymer. The composition may further comprise a hydrophilic carrier, a hydrophobic carrier, or a mixture of such carriers. The composition forms a hydrophilic but water insoluble polymeric film on the skin.

For a full understanding of the present invention, reference should now be made to the following detailed description of the preferred embodiments of the invention.

### DESCRIPTION OF THE PREFERRED EMBODIMENTS

The present invention is directed to a bioadhesive, hydrophilic. polymer film forming composition, comprising a water-insoluble graft copolymer (Copolymer), in the form of a solution, lotion, cream, film, or petrolatum or wax based preparations for treatment of mammalian skin. This invention particularly relates to the use of aqueous or non-aqueous formulations containing the Copolymer, with or without biologically active agent, on mammalian skin for cosmetic purposes and/or treatment of dermatological conditions and ailments including skin wounds. This invention also relates to a method of treatment of mammalian skin using the bioadhesive polymeric composition. This invention further relates to transdermal administration of biologically active agents for systemic activity at tissues or organs in the body other than the topical site of application on skin.

The preparation of the hydrophilic polystyrene graft copolymers has been disclosed by R. Milkovich, et al., U.S. Pat. No. 4,085,168. Intravaginal and mucoadhesive drug delivery compositions based on certain compositions of hydrophilic graft copolymers have been claimed by Shah in U.S. Pat. Nos. 5,814,329 and 5,942,243. The graft copolymers suitable for use in this invention include those described in the Shah patents referenced above. The graft copolymer has a hydrophilic polymeric main chain and a hydrophobic polymeric side chain. The main chain is comprised of hydrophilic monomeric units, some which have acidic groups. The hydrophobic side chain moiety is polystyrene. The graft copolymer is prepared by free radical initiated polymerization of a polystyrene macromonomer having an ethylenically unsaturated functional group with hydrophilic comonomers. Various graft copolymer compositions and a detailed method of preparation of such graft copolymers is described in the above referenced patents. The use levels of the graft copolymer in the compositions of this invention may range from about 0.3% to about 10%, preferably from about 0.3% to about 5%, and most preferably from about 0.3% to about 3%. The exact amount of the graft copolymer in the composition will depend upon the specific end use of the composition.

Compatible water soluble polymers may be used with the graft copolymer in the compositions of this invention. The water soluble polymer increases hydration capacity of the graft copolymer. Compatible water soluble polymers suitable for blending with the graft copolymer include, but are not limited to, poly(N-vinyl 2-pyrrolidone), hydroxypropyl cellulose, xanthan gum, hydroxyethyl cellulose, and poly(N,N-dimethylacrylamide). The proportion of the water soluble polymer used in blending may vary from 0 to 75 percent by weight, based on the combined weights of the water soluble polymer and the graft copolymer.

A simple dosage form for topical application of composition of this invention to mammalian skin is a non-aqueous solution of the Copolymer in an acceptable solvent or a mixture of solvents. The solvent may be volatile or non-volatile. Examples of volatile solvents that may be used include, but not limited to, 1-methoxy-2-propanol (Dowanol PM®) or a mixture of isopropyl alcohol and acetone. The Copolymer solution may also include a hydrophilic plasticizer, such as polyethylene glycol 400, along with an active agent. The solution may be applied to skin as a spray, a roll-on or any other convenient method. Upon topical application of the solution to skin, the solvent evaporates resulting in formation of bioadherent Copolymer film, containing the active agent, on the skin. Alternatively, solution of the Copolymer in a water-miscible non-volatile solvent, such as N-methyl 2-pyrrolidone (Pharmnasolve™, International Scientific Products), when applied to skin as a thin layer, forms a hydrogel Copolymer film upon hydration by absorption of transepidermal water loss and atmospheric moisture. Other examples of non-volatile solvents include ethoxy diglycol (monoethyl ether of diethylene glycol) and tripropylene glycol. One of the advantages of this composition is that it may be easily formulated with either hydrophilic or hydrophobic carriers, making it very easy to incorporate other ingredients, or incorporate the composition into existing product formulations, without having to make changes therein. The universal applicability of the film-forming compositions of the present invention provides a manufacturing advantage, and may also provide a regulatory clearance advantage, while increased usage will provide economies of scale.

Other dosage forms for topical application include lotion, cream, gel, or an ointment, the preparation of which is well known to those skilled in the art. Gel, lotion, or a cream would be the cosmetically preferred form for topical skin application. Lotions and creams are emulsions of oil and water, and generally contain other ingredients such as emolients, preservatives, vitamins, actives, etc. Water in oil emulsions are also suitable topical use. The Copolymer may be incorporated in the water phase by dispersing it under mechanical forces such as those produced by a homogenizer. A gel is a particularly appealing dosage form of the graft copolymer of this invention. It was unexpectedly found that although the graft copolymer is insoluble in water, it forms a very homogenous and stable gel under high energy mixing as produced by a homogenizer. The gel has a bluish haze indicating that it is not a true thermodynamic solution. Gel, cream or a lotion, when applied to skin, forms an imperceptible, bioadherent hydrogel film upon “drying”. The “drying” occurs by evaporation of volatile solvents, and/or equilibration with the skin, of non-volatile solvents and/or other ingredients. The “dry” film is very hydrophilic, and yet water insoluble.

It was unexpectedly found that the aqueous gel form of the graft copolymer has a foam stabilizing effect. Aqueous solutions of a detergent, such as that used in shampoo formulations, containing a graft copolymer of this invention forms a foam which is significantly more stable than that formed without the graft copolymer. Such gel formulations are useful in personal care detergent preparations, such as hair shampoo and shaving cream or gel. An added benefit provided by the graft copolymer is lubricity on skin and hair.

The aqueous gel form of the of the graft copolymer, when used in hair care preparations, also imparts hair moisturizing and conditioning effect by virtue of forming a thin water insoluble hydrogel polymer coating on hair.

It has been previously disclosed by Shah (U.S. Pat. No. 5,942,243) that a non-aqueous solution of the graft copolymer in a solvent such as N-methyl 2-pyrrolidone or ethoxy diglycol forms a water insoluble hydrogel in an aqueous environment. This property is very advantageous in the preparation of cosmetic formulations such as lipstick, face make up, and mascara. The graft copolymer solution, when formulated with the pigments used in such preparations, appears to encapsulate or bind the pigments upon gelation by water. Thus, a formulation comprising of a dispersion of pigments in the graft copolymer solution upon application to skin forms a bioadherent colored gel due to skin moisture. Specific cosmetic formulation (e.g. lipstick, face make up, and mascara) may contain other hydrophobic ingredients generally used in its preparation.

A preformed film of the Copolymer is an effective dosage form for application to skin wounds. The Copolymer film can be made by casting a solution of the Copolymer in a volatile organic solvent, such as methylene chloride, chloroform, 1-methoxy-2-propanol, and N,N-dimethylformamide, on a release liner and drying by evaporation of the solvent. A solution of the Copolymer in a water miscible non-aqueous solvent is another means of forming a hydrogel on skin wounds. A preformed film or the non-aqueous solution may optionally contain an active, such as an anti-microbial, anti-inflammatory, and a wound healing agent.

The topical application of any one of the foregoing dosage forms on skin is a method of forming an imperceptible hydrogel film comprising of the Copolymer. Key properties of the formed Copolymer film on skin are its transparency, hydrophilicity, moisture retentiveness, breathability, bioadhesion (high substantivity on skin), and capability to provide sustained release of an active agent, which can be as simple as moisture, over a prolonged period of time. The method of treating skin with a Copolymer dosage form of this invention provides the benefit of a long lasting treatment, which at once enhances user convenience and compliance. Further, by virtue of providing sustained release of an active to skin, the effectiveness of the active may be optimized and undesirable side effects may be decreased.

The bioadhesive Copolymer dosage forms of this invention may be employed for a variety of different uses including, but not limited to, the following:

1 Cosmetic—skin moisturizers (e.g. water, glycerine, petrolatum, dimethicone, lactic acid salts, alpha-hydroxy acids), foundations, formulations for eliminating or reducing fine lines and wrinkles, skin toners, skin moisturizers, face make up, lip color, lip treatment for eliminating fine lines and wrinkles or “puffing up” the lips, eye shadow, mascara, fragrances, deodorants, antiperspirants, sunscreen, hair care products, and cellulite treatment (e.g. delivery of xanthines such as caffeine, theophylline, and theobromine to skin).

2 Dermatological—acne treatment (e.g. benzoyl peroxide, salicylic acid, retinoic acid, and azelaic acid), steroidal anti-inflammatory drugs (e.g. hydrocortisone, triamcinolone acetonide, betamethasone valerate, betamethasone dipropionate, betamethasone benzoate, clobetasol propionate, halcinonide, desoximethasone, amcinonide, fluocinonide, and other corticosteroids), non-steroidal anti-inflammatory drugs (e.g. ibuprofen), antihistamine (e.g. benadryl, brompheniramine, and diphenhydramine), local anesthetics (e.g. lidocaine, pramoxine, and benzocaine), topical antibiotics (e.g. neomycin, bacitracin, tetracycline, erythromycin, quinolone antibacterials, and azithromycin), antifungals (clotrimazole, miconazole, and tolnafnate), antiparasitics (e.g. metronidazole), antispasmodic and anticholinergic (e.g. atropine), antiviral (e.g. acyclovir and docosanol), hair growth stimulants (e.g. minoxidil), vasodilator (e.g. alprostadil), urological drugs (e.g. oxybutynine), and an anti-psoriatic compound such as anthralin (dithranol), coal tar extract, and the like.

3 Transdermal administration of biologically active agents for systemic activity at tissues or organs in the body other than the topical site of application on skin—steroids (e.g. testosterone, estradiol, progesterone, and its conjugates), nicotine, nitroglycerine, scopolamine, oxybutynine, and fentanyl.

4. Skin Wounds for wound protection, absorption of wound exudates, autolytic wound debridement, and delivery of active agents (e.g. antimicrobials).

The bioadhesive compositions of this invention may also include an effective amount of a skin penetration enhancing agent, or pharmacologically inert substance that is capable of enhancing the penetration rate of a therapeutic agent through the skin. Preferably, the penetration enhancing agent will increase the flux rate of a therapeutic agent through the skin by altering the thermodynamic activity of a penetrant or a co-solvent incorporated into the formulation, or by affecting the partition coefficient between the therapeutic agent and the skin to promote release of the therapeutic agent and the like from the formulation into the skin. Some of the skin penetration enhancers that may be included in the compositions of this invention include, but are not limited to, dimethyl sulfoxide, N,N-dimethyl acetamide, 2-pyrrolidone, 1-methyl-2-pyrrolidone, Carbitol solvent (Union Carbide), propylene carbonate, 1,5-dimethyl-2-pyrrolidone, 2-pyrrolidone-5-carboxylic acid, oleic acid, and the like.

### EXAMPLES

### EXAMPLE 1

Synthesis of Bioadhesive Graft Copolymers

(A) Poly(N,N-dimethylacrylamide-co-acrylic acid-co-polystyrene ethyl methacrylate): In a 500-ml resin kettle equipped with a stirrer, a thermometer, a condenser, and a nitrogen inlet tube, was placed 43.75 g. of N,N-dimethylacrylamide, 5.0 g. of acrylic acid, and 1.25 g. of polystyrene ethyl methacrylate macromonomer having a number average molecular weight of 12,000 (manufactured by Polymer Chemistry Innovations, Inc.), and a mixture of 55 ml of acetone plus 10 ml of 1-methoxy-2-propanol (Dowanol PM). A solution of 50 mg of azobisisobutyronitrile in 2.0 ml of acetone was slowly added to the mixture under constant stirring until a completely clear solution was obtained. The reaction mixture was heated to 50° C. and maintained at that temperature for a period of 1 hour under nitrogen atmosphere. The reaction mixture was then further heated and allowed to reflux for an additional period of 3 hours also under nitrogen atmosphere, after which time a viscous polymer solution was obtained. A solid graft copolymer was isolated by evaporation of the solvent and any residual unreacted volatile monomeric components in a vacuum oven. The solid product was then crushed into small fragments and purified by swelling and extraction with acetone. The acetone extracted mass was then dried in a vacuum oven at 50° C. until free of solvent and residual monomer to yield 45 g of a colorless and odorless graft copolymer.

(B) Poly(N,N-dimethylacrylamide-co-acrylic acid-co-methacryloxyethyl thiocarbomoyl Rhodamine B-co-polystyrene ethyl methacrylate) (Fluorescent graft copolymer): In a 500-ml resin kettle equipped with a stirrer, a thermometer, a condenser, and a nitrogen inlet tube, was placed 21.85 g. of N,N-dimethylacrylamide, 2.5 g. of acrylic acid, and 0.625 g. of polystyrene ethyl methacrylate macromonomer having a number average molecular weight of 12,000 (manufactured by Polymer Chemistry Innovations, Inc.), 0.025 g. of methacryloxyethyl thiocarbomoyl Rhodamine B (Polyfluor 570, Polysciences, Inc.), and a mixture of 35 ml of acetone plus 7 ml of 1-methoxy-2-propanol (Dowanol PM). A solution of 25 mg of azobisisobutyronitrile in 1.0 ml of acetone was slowly added to the mixture under constant stirring until a completely clear solution was obtained. The reaction mixture was heated to 50° C. and maintained at that temperature for a period of 1 hour under nitrogen atmosphere. The reaction mixture was then further heated and allowed to reflux for an additional period of 3 hours also under nitrogen atmosphere, after which time a viscous polymer solution was obtained. A solid graft copolymer was isolated by evaporation of the solvent and any residual unreacted volatile monomeric components in a vacuum oven. The solid product was then crushed into small fragments and purified by swelling and extraction with acetone. The acetone extracted mass was then dried in a vacuum oven at 50° C. until free of solvent and residual monomer to yield 46 g of a odorless, light pink colored graft copolymer having covalently bonded fluorescent Rhodamine B moieties in its chains.

### EXAMPLE 2

Preparation of Topical Bioadhesive Formulations

(A) Polymer Solution: A 15 g. sample of the dried graft copolymer of Example 1A was dissolved in 85 g. of Pharmasolve® (N-methyl 2-pyrrolidone, International Scientific Products) to obtain a clear, colorless, viscous solution.

(B) Cosmetic Cream: A 933 g. sample of Jergens® hand lotion was placed in a 2-liter beaker and heated to 70° C. under continuous stirring. To the hot fluid mass was gradually added 67 g. of 15% solution of the graft copolymer of Example 1A in Pharmasolve® while being dispersed therein by a Silverson homogenizer. After all the solution was added and thoroughly dispersed, the resulting mixture was allowed to cool to room temperature and allowed to stand for 24 hours to yield a thick smooth cream.

(C) Cosmetic Cream Base: A cosmetic cream base was made using the following ingredients and procedure:

| | | Amount |
|---|---|---|
| INGREDIENT | | % W/W |
| (a) | Deionized water | 63.10 |
| | Propylene glycol (2) | 5.00 |
| | Disodium EDTA (3) | 0.10 |
| (b) | Carbopol 934 (Carbomer) | 0.30 |
| (c) | Cremophor ® CO 40 (PEG-40 Hydrogenated Castor Oil) | 4.00 |
| | Cremophor ® WO 7 (PEG-7 Hydrogenated Castor Oil) | 0.30 |
| | Cremophor ® GS 11 (Glyceryl Stearate) | 3.00 |
| | Lanette ® Wax 0 (Cetearyl Alcohol) | 2.00 |
| | Witoconol APM (PPG-3 Myrstyl Ether) | 10.00 |
| | Protopet White IS (Petrolatum-Vaseline) | 5.00 |
| | Liponate 1PM (Isopropyl Myristate) | 5.00 |
| | Alpha-Bisabolol, natural (Bisabolol) | 0.20 |
| (d) | 50% aqueous solution of Triethanolamine | 1.00 |
| (e) | Germaben ® II (Propylene Glycol & Diazolidinyl Urea & | 1.00 |
| | Methyl Paraben & Propyl Paraben) (ISP) | |

PROCEDURE: Group (a) ingredients were first mixed together by means of a propeller type mixer. Then, Carbopol (b) was sprinkled on to it under continuous agitation and mixed for an additional 30 minutes to fully hydrate the Carbopol and form the aqueous phase.

Group (c) ingredients were combined together and preheated to 80° C. under stirring to form the oil phase. The preheated oil phase was then gradually added to the aqueous phase, which was also preheated to 80° C., while being continuously dispersed by means of Silverson homogenizer. The triethanolamine solution(d) was then added and homogenized with the hot fluid mixture. The mixture was then allowed to cool to 45° C., the Germaben® II (e) was added, mixed in it. Upon cooling the mixture to room temperature, a smooth lotion was formed.

(D) Antiviral Docosanol Cream with Graft Copolymer

A 7 g. of the graft copolymer solution of Example 2A was preheated to 70° C. and gradually added to 83 g. sample of the cosmetic cream base of Example 2C, also preheated to 70° C., under continuous homogenization. Then, 10 g. of docosanol was added to the mixture and homogenized. After the mixture was cooled to room temperature and allowed to stand for 24 hours, it formed a thick, smooth, white cream.

(E) Fluorescent Cosmetic Cream

A 93 g. sample of cream base of Example 2C was placed in a 250-ml beaker and heated to 70° C. under continuous stirring. To the hot fluid mass was gradually added 7 g. of 15% solution of the graft copolymer of Example 1B in Pharmasolve® while being dispersed therein by a Silverson homogenizer. After all the solution was added and thoroughly dispersed, the resulting mixture was allowed to cool to room temperature and allowed to stand for 24 hours to yield a thick smooth cream.

### EXAMPLE 3

Substantivity of Graft Copolymer Cream on Skin

The cosmetic cream of Example 2E, which contained graft copolymer covalently tagged with a fluorescent moiety, was spread onto the epidermal surface of a dermatomed human cadaver skin (obtained from New York Fire fighters Skin Bank). The cream was allowed to air dry on the skin forming a virtually imperceptible layer. Shining long wavelength ultraviolet light on the skin under darkness revealed uniform pink fluorescence all throughout, where the cream was applied.

The skin was then kept under running tap water for 1 minute and then submerged in plain water for an additional period of 15 minutes. After this treatment the skin was removed from the water. Once again upon illuminating the water treated skin with long wavelength ultraviolet light under darkness revealed uniform pink fluorescence all throughout, where the cream was applied. There was no apparent decrease in the intensity of fluorescence by water treatment. On the basis of these observations, it may be concluded that the graft copolymer film formed on the surface of the cadaver skin showed a high degree of bioadhesion to the skin and offered a substantial resistance to dislodgment by water although the graft copolymer was very hydrophilic.

### EXAMPLE 4

Human use Test of Non-Aqueous Graft Copolymer Solution

An aliquot of the graft copolymer solution of Example 2A was spread with fingers on the back of the hand of a human volunteer. This solution became tack-free on the hand in a period of about 15 minutes. The site of application was tested periodically by applying a few drops of water to it. The presence of the graft copolymer was detected by the perception of gel like slippery feel to the touch. Upon such testing, the presence of the graft copolymer on the skin was evidenced for a period of at least 24 hours.

### EXAMPLE 5

Clinical Evalaution of Skin Moisturization by Graft Copolymer Cosmetic Cream

Control Product: Jergens® lotion.

Test Product: Cosmetic cream of Example 2A, which was prepared from Jergens® lotion and graft copolymer.

Instrumentation: Corneometer—CM 820

Test Method: 14 women volunteer subjects, who met the inclusion/exclusion criteria, were impaneled. Panelists were instructed to discontinue the use of all moisturizing products on the arms for a 7-day conditioning period. After this period, the subjects returned to the laboratory and were required to acclimate to ambient temperature and relative humidity for 30 minutes. Triplicate baseline Corneometer measurements were taken from the right and left forearms of each subject and recorded to indicate the level of skin moisturization.

Each test material was randomly assigned for treatment of either the left or right volar surface of the forearm. Each test material was then applied to the designated forearm by the individual panelists. Triplicate Corneometer readings were taken from each forearm at 0, 2, 4, 6, and 24 hours after application of the formulations.

Results

No adverse reactions were reported.

Percentage Mean Differences in Corneometer Measurements from the Baseline:

| | | | % Increase in |
|---|---|---|---|
| | Cream of | | Skin Moisturization |
| Time | Example 2A | Control | Over Control |
| 0 Hrs | 68 | 68 | 0 |
| 2 Hrs | 17 | 10 | 70 |
| 4 Hrs | 14 | 8 | 75 |
| 6 Hrs | 9 | 5 | 80 |
| 24 Hrs | −1 | −2 | 0 |

These results indicated an average of 75% increase in skin moisturization, by the graft copolymer containing cosmetic cream of Example 2A, with respect to that by the control for a period of at least 6 hours.

### EXAMPLE 6

Human use Test of Antiviral Docosanol Cream Containing Graft Copolymer

Lip surfaces of a male human volunteer, who was infected with herpes simplex virus (HSV I) as indicated by a “fever blister” on the lip, were treated once/day with the docosanol cream of Example 2D containing the graft copolymer. The fever blister of the volunteer healed within a period of 3-4 days. It was the experience of the volunteer that on other occasions, when he had treated the blisters 3-4 times each day with commercial products, the healing time was 5 days or more.

### EXAMPLE 7

Wound Treatment with Non-Aqueous Graft Copolymer Solution

A bleeding skin surface wound of a Human volunteer was treated with a gauze pad soaked with the non-aqueous graft copolymer solution of Example 2A. The gauze pad was kept in place on the wound by support from a convention bandage. The gauze pad was removed after a period of 48 hours. The non-aqueous graft copolymer solution had become a soft hydrogel covering the wound by absorption of wound exudates.

### EXAMPLE 8

Anti-Cellulite Gel Formulation

A cosmetic gel was made using the following ingredients, including those known to be effective in fat burning and reduction of the appearance of cellulite upon topical application, and procedure:

| | | Amount |
|---|---|---|
| INGREDIENT | | % W/W |
| (a) | Graft copolymer of Example 1A | 1.6 |
| | Xanthan Gum | 0.4 |
| | Water | 70.0 |
| | Alcohol | 15.0 |
| | Ethoxy Diglycol | 10.0 |
| (b) | Theophylline | 1.0 |
| | Carnitine | 1.0 |
| | Caffeine | 1.0 |

PROCEDURE: Group (a) ingredients were first mixed together by means of a homogenizer to form a clear flowing gel having a slight bluish haze. Then, the group (b) ingredients were added to it and stirred by means of a propeller type mixer to form a homogenous solution.

The anti-cellulite gel thus prepared could be easily applied and spread over skin to form an imperceptible and invisible hydrogel film, containing the active ingredients of the group (b).

### EXAMPLE 9

Foam Stabilization by Graft Copolymer

Following two solutions (a) and (b), with and without the graft copolymer of Example 1A, were prepared.

(a) A small amount, 0.185 grams, of the graft copolymer of Example 1A and 8 grams of a 70% aqueous solution of sodium laureth sulfate were uniformly mixed in 31.815 grams of water by means of a homogenizer.

(b) An aliquot, 8 grams, of 70% aqueous solution of sodium laureth sulfate were mixed in 32 grams of water by means of a propeller type mixer.

Foam produced by mixing & agitating equal amounts of water with solution (a), containing the graft copolymer, persisted for a much longer duration than that produced in a similar manner by the solution (b).

There has thus been shown and described a novel bioadhesive, hydrophilic composition for treating mammalian skin which fulfills all the objects and advantages sought therefore. Many changes, modifications, variations and other uses and applications of the subject invention will, however, become apparent to those skilled in the art after considering this specification which discloses the preferred embodiments thereof. All such changes, modifications, variations and other uses and applications which do not depart from the spirit and scope of the invention are deemed to be covered by the invention, which is to be limited only by the claims which follow.

1. A film-forming composition for application to mammalian skin, said composition comprising,
a) from about 0.3% to about 10% by weight of the total composition, of a graft Copolymer, comprising a hydrophilic polymer main chain comprising monomeric units, some of which have acidic groups, and a hydrophobic polymeric side chain comprising polystyrene;
b) from about 0 to about 75% of water soluble polymer by weight, based on the combined weights of the water soluble polymer and the graft Copolymer; said composition further being comprised of hydrophilic or hydrophobic carriers or a mixture of the same; and said composition forming a hydrophilic but water insoluble polymeric film on the skin.

2. The film-forming composition of claim 1, wherein said composition comprises from about 0.3 to about 5% by weight of the graft Copolymer.

3. The composition of claim 1, comprising from about 0.3% Copolymer to about 3% Copolymer.

4. The composition of claim 1, as a solution, emulsion, dispersion, lotion, cream, film, or petrolatum or wax based preparation.

5. The film-forming composition of claim 1, wherein said composition is a non-aqueous formulation.

6. The film-forming composition of claim 1, further comprising a biologically active agent.

7. The film-forming composition of claim 4, further comprising a biologically active agent.

8. A method of treatment of mammalian skin comprising applying to the said skin, an effective amount of a composition of claim 1.

9. A skin moisturizer comprising the aqueous formulation of claim 1.

10. The method of claim 8, wherein the method of applying the composition is selected from the group consisting of a spray, a roll-on, immersion, dipping, applying by brush, or spattering.

11. A stable gel, comprising the homogenized formulation of
a) from about 0.3% to about 10% by weight of the total composition, of a graft Copolymer, comprising a hydrophilic polymer main chain comprising monomeric units, some of which have acidic groups, and a hydrophobic polymeric side chain comprising polystyrene;
b) from about 0 to about 75% of water soluble polymer by weight, based on the combined weights of the water soluble polymer and the graft copolymer; in an aqueous formulation, said composition forming a hydrophilic but water-insoluble polymeric film on the skin.

12. A foam stabilizer, comprising the composition of claim 1.

13. A detergent comprising the foam stabilizer of claim 12.

14. A shampoo comprising the foam stabilizer of claim 12.

15. A hair conditioner comprising the composition of claim 1.

16. A method of treatment of mammalian skin, comprising a transdermal, sustained release of a biologically active agent from the film-forming composition comprising,
a) from about 0.3% to about 10% by weight of the total composition, of a graft Copolymer, comprising a hydrophilic polymer main chain comprising monomeric units, some of which have acidic groups, and a hydrophobic polymeric side chain comprising polystyrene;
b) from about 0 to about 75% of water soluble polymer by weight, based on the combined weights of the water soluble polymer and the graft copolymer;
c) an effective amount of the biologically active agent; said composition further being comprised of hydrophilic or hydrophobic carrier or a mixture of the same; and said composition forming a hydrophilic but water insoluble polymeric film on the skin.

17. A method of systemic treatment of mammal tissue, comprising a transdermal, sustained release of an active agent from the film-forming composition comprising,
a) from about 0.3% to about 10% by weight of the total composition, of a graft Copolymer, comprising a hydrophilic polymer main chain comprising monomeric units, some of which have acidic groups, and a hydrophobic polymeric side chain comprising polystyrene;
b) from about 0 to about 75% of water soluble polymer by weight, based on the combined weights of the water soluble polymer and the graft copolymer;
c) an effective amount of the biologically active agent; said composition further being comprised of hydrophilic or hydrophobic carrier or a mixture of the same; and said composition forming a hydrophilic but water insoluble polymeric film on the skin.

18. A face make up, comprising the composition of claim 1.

19. A lipstick comprising the composition of claim 1.

20. A mascara comprising the composition of claim 1.
